(19) United States
(12) Patent Application Publication
(10) Pub. No.: US 2002/0164790 A1
(43) Pub. Date: Nov. 7, 2002

(54) Lung stem cells and lung regeneration

(76) Inventors: David Warburton, La Canada, CA (US); Barbara Driscoll, Los Angeles, CA (US); Susan A. Buckley, Pasadena, CA (US); Margaret A. Schwarz, La Canada-Flintridge, CA (US)

Correspondence Address: MYERS BIGEL SIBLEY & SAJOVEC, PO BOX 37428, RALEIGH, NC, 27627, US

(21) Appl. No.: 09/732,163

(22) Filed: Dec. 7, 2000

Related U.S. Application Data
(60) Provisional application No. 60/169,545, filed on Dec. 7, 1999.

(51) Int. Cl. A61K 48/00; C12N 5/08

(52) U.S. Cl. 435/366; 424/93.7

(57) ABSTRACT

A method of stimulating the growth of lung alveolar surface in a lung comprises providing progenitor or stem cells capable of regenerating lung alveolar surface, and then administering the progenitor or stem cells to the lung in an amount sufficient to stimulate the growth of lung alveolar surface therein.

### RELATED APPLICATIONS

This application claims the benefit of commonly owned, copending provisional patent application Serial No. 60/169,545, filed Dec. 7, 1999, the disclosure of which is incorporated by reference herein in its entirety.

### GOVERNMENT SUPPORT

This invention was made with Government support under Grant Nos. HL44060, HL60231 and HL44977 from the National Institutes of Health. The U.S. Government has certain rights to this invention.

### FIELD OF THE INVENTION

The present invention concerns methods and compositions useful for facilitating lung regeneration, both in vivo and in a lung that is being transplanted from a donor to a recipient.

### BACKGROUND OF THE INVENTION

Congenital diaphragmatic hernia (CDH) occurs in about 1 in 3000 human live births. Although it is associated with several genetic defects, its exact etiology is not known. Newborns with CDH have a 40-50% mortality, which is primarily caused by the associated pulmonary hypoplasia. The hypoplastic lungs are not capable of providing adequate gas exchange for oxygenation, and persistent pulmonary hypertension leads to refractory hypoxia (right to left shunting). Unlike other causes of neonatal respiratory failure, infants with CDH are often unresponsive to the modern therapeutic armamentarium, because it does not solve the basic problem of lung hypoplasia (The baud et al. (1998) Biol. Neonate 74:323-336).

The hypoplastic lung in CDH is developmentally delayed. There is a marked reduction from 21 generations of airways in the normal human lung, to 12-14 generations in the psi-lateral and 16-18 generations in the contra-lateral lung in CDH (Areechnon and Reid (1963) Br Med J 1:230-33). There is also a delay in the differentiation of alveolar epithelial cells with a resultant surfactant deficiency (Wilcox et al. (1996) Clin Perinatol 23:771-779), and fewer and more arterialized vascular branches (O'Toole et al. (1996) Clin Perinatol 23:781-794). In addition to the effect of mechanical compression by the herniated abdominal viscera, lung hypoplasia in CDH may also result from a primary abnormality in airway branching (Jesudason et al. (2000) J Pediatr Surg 35:124-7; Keijzer et al. (2000) Am J Path 156:1299-1306). However, the molecular mechanisms underlying lung hypoplasia in human CDH have not been fully investigated.

Since the first description of Nitrofen-induced diaphragmatic hernias in rodents by Iritani in 1984, the murine nitrofen-induced model of CDH has been extensively studied, and by now is widely accepted as a well-established model that has many phenotypic similarities to the human condition (Iritani (1984) Anat Embryol 169:133-9; Greer et al. (2000) Pediatric Pulmonol 29:394-9; Kluth et al. (1990) J Pediatr Surg 25:850-4). Using this model in mice, it has been shown that Nitrofen causes primary pulmonary hypoplasia, which is worsened by the presence of a hernia (Coleman et al. (1998) Am J Physiol 274:636-646). In rats, nitrofen has also recently been shown to reduce branching morphogenesis before diaphragmatic closure, both in vitro and in vivo (Keijzer et al. (2000) Am J Path 156:1299-1306). Since Nitrofen-exposed embryonic lungs are clearly hypoplastic prior to the appearance of an actual diaphragmatic defect, an evaluation of candidate factors known to be required for early lung development was initiated (Warburton et al. (2000) Mech Dev. 92:55-81).

During mouse lung morphogenesis, the distal mesenchyme has long been known to regulate the growth and branching of the adjacent endoderm through the secretion of soluble factors (recently reviewed by Warburton et al, 2000 (Mech Dev. 92:55-81). Bellusci et al (Bellusci et al. (1997) Development 124:4867-78) reported that FGF 10 is a mesenchyme-derived factor that plays a critical role in patterning the early branching events in lung development. Fgf10 null mutant mice and transgenic mice expressing dominant negative forms of the FGF10 receptor, Fgfr2-IIIb, have a dramatic inhibition of bronchial branching (Min et al. (1998) Genes Dev 12:3156-61; Peters et al. (1994) EMBO J 13:3296-3301). Fgf10 is expressed in a temporospatially specific pattern in the peripheral embryonic lung mesenchyme near the positions where primary, secondary and tertiary bronchi bud (Bellusci et al. (1997) Development 124:4867-78). The buds grow towards these areas of Fgf10 expression. Thus Fgf10 appears to stimulate and direct early bronchial branching. FGF-pathway signaling is modified at each stage of branching by genetic feedback controls. Sonic hedgehog (Shh), which is strongly expressed in the distal epithelium, may function as a negative signal for Fgf10 (Bellusci et al. (1997) Development 124:53-63; Grindley et al. (1997) Dev Biol 188:337-348). Shh inhibits Fgf10 expression in the mesenchyme near growing tips, where the initial Fgf10 expression domain splits laterally into two domains. Two new buds then sprout, each targeting one of the lateral subdomains of Fgf10 expression. Mice in whom Shh has been inactivated also have profound impairments of lung branching (Pepicelli et al. (1998) Curr Biol 8:1083-1086). Other key antagonists of the FGF-pathway include members of the Sprouty gene family. Murine Sprouty 2 (mSpry2) is an inducible negative regulator of FGF receptor tyrosine kinase signaling that is expressed in the distal epithelium of the embryonic mouse lung, adjacent to the mesenchymal loci of Fgf10 expression, at embryonic stages when lung epithelial buds are highly responsive to FGF10. Abrogation of mSpry2 expression in lung organ cultures with antisense oligonucleotides increases branching morphogenesis and surfactant gene expression (Teffi et al. (1999) Curr Biol 9:219-22).

Alveolar epithelial type 2 cells (AEC2) have been designated the primary progenitor cell of the alveolar epithelium (Ten Have-Opbroek (1979) Dev. Biol. 69:408-423). In the embryo, AEC2 arise from multipotent stem cells which line the primitive respiratory tract. These primitive, proliferative embryonic epithelial precursors co-express several markers, including SP-A, SP-C, CC.10 and cGRP, which are subsequently expressed in separate, differentiated lineages in the mature fetus and in the adult, including AEC2, Clara cells and pulmonary neuroendocrine cells (Wuenschell et al. (1996) J Histochem. Cytochem. 44:113-123). At late gestation, the AEC lineage becomes restricted, such that only AEC type 1 and type 2 cells are produced (Mason et al. (1997) Am. J Respir. Cell Mol. Biol. 16:355-363). Type 2 cells manufacture surfactant and can differentiate, as required, into AEC1 (Ten Have-Opbroek, et al. (1991) Anat. Rec. 229:339-354). AECI are terminally differentiated, incapable of dividing, and perform the necessary lung function of gas exchange. However, the ability to divide must be retained by a sub-population within the lung alveolar epithelium throughout the life span of any animal, in order to replace damaged cells (Adamson and Bowden (1974) Lab Invest. 30:35-42; Evans, et al. (1975) Exp. Mol. Pathol. 22:142-150). This stem or progenitor cell function has been ascribed to AEC2.

Recent studies have shown that the ability of a cell to divide over an indefinite life span may require the expression of telomerase, a ribonucleoprotein which stabilizes the telomeres of chromosomes in actively growing cells (Blackburn, et al. (1989) Genome 31:533-560). Telomerase contains structural features and activity similar to those of reverse transcriptases, such that the catalytic subunit of the enzyme has been termed Telomerase Reverse Transcriptase (TERT). Its structure and function are highly conserved among species. Human telomerase and its mouse orthologue, termed hTERT and mTERT respectively, have recently been cloned and characterized (Greenberg, et al. (1998) Oncogene 16:1723-17; Harrison and Lerner (1991) Blood 78:1237-40; Kilian, et al. (1997) Hum. Mol Genet. 6:2011-2019; Meyerson, et al. (1997) Cell 90:785-795; Nakamura, et al. (1997) Science 277:955-959). In cells undergoing terminal differentiation, telomerase activity is down regulated, and chromosomal telomeres become progressively shorter (Greider (1998) Current Biol. 8:R178-R181). Multiple studies have shown that in normal adult tissues, telomere length and telomerase activity appear to correlate well with the differentiation stage of a cell, as well as its potential to act as a stem cell upon appropriate stimulation (Greider (1998) Current Biol. 8:R178-R181; Lavker, et al. (1993) J Invest. Dermatol. (Suppl.) 101:16S-26S; Mason et al. (1997) Am. J Respir. Cell Mol. Biol. 16:355-363).

The self-renewing population of progenitor cells found in most tissues have been termed stem cells. Telomerase expression correlates with self-renewal potential in many cell types, including epithelial cells (Morrison, et al. (1997) Cell 86:287-298; Yasumoto et al. (1996) Oncogene 13:433-439). Unlike tumor cells, stem cells are not immortal, and show decreasing telomere length with increasing age (Morrison, et al. (1996) Nature Med. 2:202-206; Vaziri, et al. (1994) Proc. Nat. Acad. Sci. USA 91:9857-9860). Thus, telomerase may regulate self-renewal capacity by reducing the rate at which telomeres shorten. Understanding the function of telomerase in mediating self-renewal and survival of AEC stem cells would be a major advance in AEC biology, since a putative AEC stem or progenitor cell could be the main source of epithelial expansion during development, of epithelial repair following injury, and possibly of alveolar adenocarcinoma.

### SUMMARY OF THE INVENTION

A method of stimulating the growth of lung alveolar surface in a lung in need thereof comprises the steps of: providing progenitor or stem cells capable of regenerating lung alveolar surface; and administering said progenitor cells to said lung in an amount sufficient to stimulate the growth of lung alveolar surface therein.

In one embodiment, the lung is in vivo in a subject in need of said treatment.

In another embodiment, the lung has been removed from a donor and the administering step is carried out ex vivo, and the administering step is followed by the step of transplanting that lung into a recipient in need thereof.

Further aspects of the present invention include pharmaceutical formulations comprising progenitor and/or stem cells in combination with a pharmaceutically acceptable carrier therefore, as well as the use of such formulations for carrying out the methods described above.

### BRIEF DESCRIPTION OF THE DRAWINGS

FIG. 1 shows Fgf10 expression in control and nitrofen-exposed mouse embryonic lung detected by whole-mount in situ hybridization at E13.5. Note high levels of Fgf10 expression present in the mesenchyme adjacent to the epithelial buds of control lungs (A) and at higher magnification in the left lobe of a control lung (C), correlating with the sites of future dichotomous branching. The hypoplastic nitrofen-exposed lung shows profound disturbances in the spatio-temporal expression of Fgf10 (B, C, D, E, F). Fgf10 transcripts are detected only in the caudal part of the left lobe (F), Fgf10 expression was nearly totally abolished in a more severe hypoplastic left lobe (E).

FIG. 2 shows Spry2 expression in control and nitrofen-exposed mouse embryonic lung detected by whole-mount in situ hybridization. At E12.5, Spry2 expression is slightly less expressed in the distal tips of the epithelium (A, E). Spry2, localized to the distal tips of the epithelial buds, is equally expressed in control (B, D) and nitrofen-exposed lungs (C, F, G, H) at E14.5.

FIG. 3 shows fibroblast growth factor 10 (Fgf10) mRNA expression in Control vs. Nitrofen with and without exogenous Fgf-10 (500ng/ml). The mean ratio of Fgf10 expression relative to β-actin was 2.6 in right and 0.9 in left wild type lungs. These levels of expression decreased respectively to 0.04 (right) and 0.3 (left) following in-utero exposure to nitrofen (all p<0.05). (Results shown are all corrected as ratios to β-actin mRNA.)

FIG. 4 shows Murine Sprouty 2 (mSpry2) mRNA Expression in Control vs. Nitrofen with and without exogenous Fgf-10 (500 ng/ml) (n=3 for each sample). Levels of mSpry2 mRNA expression increased by 4- to 10-fold in the presence of exogenous Fgf10 under all conditions examined. (Results shown are all corrected as ratios to β-actin mRNA.)

FIG. 5 shows that FGF-10 increases branching morphogenesis in control lungs. A. Right lung control (average of 55.7 terminal branches). B. Left lung control (average of 31.2 terminal branches). C. Right lung with FGF-10 (500 ng/ml) (average of 63.2 terminal branches). D. Left lung with FGF-10 (500 ng/ml) (average of 39.9 terminal branches).

FIG. 6 shows that FGF-10 rescues the Nitrofen-induced lung hypoplasia on the right side. A. Nitrofen-exposed right lung (average of 43.2 terminal branches). B. Nitrofen-exposed left lung (average of 11.1 terminal branches). C. Nitrofen-exposed right lung with FGF-10 (500 ng/ml) (average of 52.9 terminal branches). D. Nitrofen-exposed left lung with FGF-10 (500 ng/ml) (average of 19.7 terminal branches).

FIG. 7 shows branching morphogenesis in Control vs. Nitrofen-exposed lungs with and without Fgf-10. A. Right control lungs show a 13% increase in branching with exogenous Fgf-10 (p<0.01). B. Right nitrofen-exposed lungs demonstrate a 22% increase in branching (p<0.01). C. Left control lungs show a 28% increase with exogenous Fgf-10 (p<0.01). D. Left nitrofen-exposed lungs demonstrate a 77% increase in branching (p<0.01).

FIG. 8 shows telomerase expression in mouse lung development. Lungs were fixed, paraffin embedded and sectioned, then analyzed for mTERT expression by immunostaining. Positively stained cells appear red, due to binding of the antibody activated chromagen, AEC. In lungs from animals at gestational age E18 (E 18) and at the first hour following birth (D 0) many mTERT positive cells can be observed throughout the developing lung epithelium. This expression pattern decreases during the neonatal period. By day six (D 6), most telomerase expression staining was observed in small patches at the surface of the lung, and by day nine (D 9) only one or two mTERT positive epithelial cells per field were observed. For all panels, magnification was 200X.

FIG. 9 shows telomerase expression in hyperoxic lung injury and repair. Whole lungs from adult rats subjected to hyperoxia for 48 hours, then allowed to recover for 48 hours in room air were obtained along with lungs from control animals which had breathed room air throughout the treatment period. Lung tissue was fixed, paraffin embedded and sectioned, then subjected to immunohistochemical analysis for rTERT expression. Control animals exhibited almost no TERT expression in lung epithelium (top panel). However, lungs from animals treated with hyperoxia, then allowed to recover for 48 hours, showed a marked increase in rTERT expression (lower panel). For both panels, magnification was 200X.

FIG. 10 shows telomerase expression in fetal and adult AEC2. AEC2, isolated by standard methods from fetal rats (gestation day E21), from adult rats subjected to hyperoxia for 48 hours then allowed to recover in room air for 48 hours, as well as control adult rats, which breathed room air for the same period, were placed in culture on plastic for 24 hours, then fixed and analyzed for expression of telomerase and PCNA. Since the telomerase antibody was a rabbit polyclonal, normal rabbit serum (NRS) was used as a negative control for staining. Cells from both the fetal and hyperoxia treated animals exhibited strong expression of both telomerase and PCNA, though in each case, staining was somewhat heterogeneous. Cells from control adult animals exhibited scattered PCNA staining, and low levels of telomerase expression.

FIG. 11 shows telomerase activity in fetal and adult AEC2. A. AEC2 were isolated by standard methods from fetal rats (gestation day E21), and from adult rats subjected to hyperoxia for 48 hours (no recovery), as well as control adult rats, which breathed room air for the same period. Cells were cultured for 24 hours in DMEM/10% FBS before harvesting. Lysates were prepared and protein quantities measured such that 80 ng of protein from each lysate was included in each sample. Duplicate samples were heat treated to provide a control for heat-tolerant PCR contaminants. These samples were loaded into lanes 1, 3, and 5. Lanes 2, 4, and 6 contain the results of the TRAP assay for the unheated samples. Thus, lanes 1 and 2 contain 80 ng each heat treated and untreated control adult rat AEC2 lysate respectively, lanes 3 and 4 contain 80 ng each heat treated and untreated hyperoxia treated adult rat AEC2 lysate respectively, and lanes 5 and 6 contain 80 ng each heat treated and untreated fetal rat AEC2 lysate respectively. B. The number of telomeric repeats (TR) present in each sample was correlated to ladder bands by the formula TR=n+3, where n=the number of bands in each ladder, and TR is taken in this assay as a reflection of relative telomerase activity. By this calculation, the fetal samples produced the largest number of telomeric repeats, where the average TR was 24. The samples from the control animals contained the fewest number (average TR=5). Reflecting increased telomerase activity, the number of repeats was increased by treatment of adult animals with oxygen (average TR=18). For the fetal TR average, n=4, while for the adult averages, n=6. Values represent mean +/−SD. Using Student's t-test, the differences between control and hyperoxic (*P<0.005) and between control adult and fetal (**P<0.001) cells were determined to be significant.

### DETAILED DESCRIPTION OF THE INVENTION

The present invention concerns the use and/or stimulation of pulmonary epithelial and vascular stem/progenitor cells to induce lung regeneration in vivo in humans, as well as in lung lobes both before and after transplantation of one or both lungs from a donor to a recipient.

An object of the present invention is to exploit the regenerative properties of pulmonary epithelial and vascular stem/progenitor cells to regenerate sufficient alveolar surface area to sustain life in humans with lung failure due to pathologic causes such as emphysema, lung hypoplasia and other severe lung diseases.

The present invention is concerned primarily with human subjects, but may be practiced on other mammalian subjects such as dogs and cats for veterinary purposes.

The term “lung” as used herein refers to a complete lung, as well as a lung portion or lobe.

Stem/progenitor cells exist in the distal lung and can regenerate both alveolar epithelium and capillaries. The present invention exploits the properties of the stem cells by stimulating them to divide and differentiate in a coordinated manner, using soluble growth factors and other suitable growth factors. The stem/progenitor cells are preferably from the same species as the subject recipient, and may be obtained from the subject itself (i.e., are autologous cells).

Any growth factor capable of stimulating the growth of stem or progenitor cells may be used to carry out the present invention. Numerous growth factors are known. See, e.g., B. Alberts et al., Molecular Biology of the Cell, pg. 894 Table 17-2 (3d Ed. 1994). Such growth factors include, but are not limited to, fibroblast growth factor (or “FGF”), including all family members thereof, particularly FGF 7 and FGF 10, epidermal growth factor (or “EGF”), including all family members thereof, platelet-derived growth factor (or “PDGF”), and retinoic acid and its derivatives. Such growth factors may be used either singly or in combination.

Exogenous stem cells for use in administering to subjects are either created by nuclear transfer of the recipients own genetic material into embryonic stem cells, or collected either from autologous bone marrow, lung biopsy or from endobronchial lavage. The cells are then amplified in culture using the growth factors mentioned above to stimulate the growth thereof. Appropriate genes may then be introduced to correct genetic defects, provide targeting information and/or to optimize growth and differentiation. The cells may then be re-implanted intra-vascularly, or intra-bronchially in any suitable physiologically acceptable carrier, such as a fluorocarbon vehicle or physiological saline solution. Implantation may be done in vivo or in a lung or lobe that has been isolated from a donor and is about to be implanted into a subject.

The dosage of cells administered to the recipient lung or subject will depend upon the efficiency of uptake, the size and condition of the recipient lung or subject, etc. In general, the dosage of cells may be from 100,000 or 200,000 cells to 10 million or 100 million cells, or more.

One embodiment of a method for inducing lung regeneration by autologous stem cell replacement is as follows:

(1) Nuclear transfer of the patient's own nucleus into human embryonic stem cells; then

(2) Amplification of the homologous embryonic stem cell line; and

(3) Gene insertion, homologous recombination, or other genetic manipulation of the embryonic stem cell line to correct genetic defects, or activate stem cells to form particular lineages or to target the stem cells to particular tissues such as lung epithelium or endothelium (amplification step 2 may precede, follow, or both precede and follow step 3); then

(4) Injection of the hologous stem cells intraveneously or intra-arterially or trans-tracheally into the subject;

(5) Injected cells take up position in host tissue and initiate homologous tissue regeneration based on endogenous cues from host tissue, as well as administered growth factors or peptide mimetics such as FGF family peptides, particularly FGF10.

The present invention is explained in greater detail in the following non-limiting Examples.

### EXAMPLE 1

Novel Mechanisms in Murine Nitrofen-Induced Pulmonary Hypoplasia:

FGF10 Rescue in Culture

Materials and Methods—Nitrofen-exposed lungs. Timed-pregnant Swiss-Webster mice (Simonsen Laboratories, Gilroy, Calif.) were gavage-fed Nitrofen (2,4-dichlorophenyl-p-nitrophenyl ether; Radian International, Austin, Tex.) 25 mg on day 8 of gestation (presence of a vaginal plug=day 0). Control animals received olive oil. Using aseptic technique, the mouse embryos were harvested by cesarean section, on embryonic day 12 (E12). On retrieval, embryos were transferred to an isotonic Hank's balanced salt solution cooled on ice. They were then microdissected from their extra-embryonic membranes and using a stereomicroscope and microsurgical instruments the lungs were excised and the right and left lobes separated and placed in Hank's balanced salt solution. The University of Southern California Institutional Animal Care and Use Committee approved the use of animals in this study.

Whole Mount In Situ Hybridization. The whole-mount in situ hybridization technique was based on that previously described by Sasaki et al (Sasaki and Hogan (1995) Development 9:2105-2116). The following murine cDNAs were used as templates for synthesizing digoxin-labeled riboprobes: 584 bp FGF-10 and 948 pb full-length mouse Spry2. In order to provide a qualitative comparison of levels of gene expression between control and nitrofen-exposed lungs between E12 and E15, lungs were fixed and processed under the same conditions with respect to probe concentration and specific activity, washed at the same temperature and stringency. Photomicrographs were taken with the same exposure time.

Organ culture. E12 lungs were cultured at the air-fluid interface by placing them on 0.8 μm MF-Millipore filters (Millipore, Bedford, Mass.), supported by stainless-steel grids in culture dishes containing BGJb medium (Gibco, Grand Island, N.Y.) supplemented with 1 mg/ml ascorbic acid and 50 units/ml penicillin-streptomycin. FGF-10 was added to the culture medium at a concentration of 500 ng/ml (R&D Systems, Minneapolis, Minn. 55413). FGF10 is not very bioactive and dose response curves have demonstrated that at 500 ng/ml we see the maximum amount of branching morphogenesis. Organ cultures were maintained at 37° C. in 100% humidity, 95% air, 5% carbon dioxide for 4 days with medium changed after 2 days.

Branching morphogenesis. Branching morphogenesis was quantified by counting the number of terminal branches visible around the periphery of each lung. This was performed before and after 4 days in culture using transillumination to visualize structures, and photomicrography to record permanent images (Warburton et al, 1992).

RNA extraction and reverse transcription. Individual cultured explants were homogenized by repeated pipetting in 4 M guanidium isothiocyanate. Total RNA was then extracted using the Rapid Total RNA Isolation Kit (5 Prime □ 3 Prime, Boulder, Colo.). Reverse transcription (RT) was performed by incubating samples of individual lung RNA at 37° C. in 10 mM Tris (pH 8.4), 50 mM KCl, 2 mM MgCl2, 1 mM dithiothreitol, 5 units ribonuclease inhibitor, 0.5 mM dNTP, 100 pmol oligo (dT)12-18, and 200 units of MMLV reverse transcriptase (USB, Cleveland, Ohio). The reaction was terminated by heating for 5 minutes at 100° C. Reverse-transcribed products were then used for competitive PCR.

Competitive PCR. PCR amplification was performed using a DNA Robocycler (Stratagene, La Jolla, Calif.) with an initial denaturation at 94° C. for 3 minutes followed by thirty-five cycles of denaturation at 93° C. for 2 minutes, annealing at 62° C. for 2 minutes, and extension at 72° C. for 2 minutes. The final cycle concluded with a 5 minute extension step. The reaction mixture contained 10 mM Tris (pH 8.4), 50 mM KCl, 2 mM MgCl2 (optomized, 0.01% Trition X-1000, 20 pmol primer sets, 100 uM deoxynucleotide triphosphate, and 0.5 units Taq thermostable DNA polymerase (Promega, Madison, Wis.). A reaction mixture, containing 1 pg/μl of the appropriate competitor DNA, was added to reverse-transcribed samples derived from 50 ng of total RNA. The concentration of cDNA standard solutions was determined spectrophotometrically by absorbance at 260 nm. The equations drawn from the linear regressions for each of the standard curves were used to interpolate the mRNA amounts from their respective cDNA equivalents in each lung sample. To control for potential variations due to the efficiency of RNA extraction and RT, β-actin mRNA was also quantified in the same samples.

Competitive RT-PCR Quantification. The same primers for mouse mSpry2 were used to amplify both the cDNA and competitor for each gene of interest. The upstream primer of cDNA synthesis was 5′-TGTGAGGACTGTGGCAAGTGC-3′ (SEQ ID NO: 1) and the downstream primer was 5′-TTTAAGGCAACCCTTGCTGG-3′ (SEQ ID NO:2) resulting in a 300 bp PCR product. Two composite primers were synthesized to construct the mSpry2 competitor. Each set of composite primers contained the mSpry sequence as well as a short sequence designed to hybridize to the cDNA of interest. This allowed the incorporation of the mSpry2 sequence into the DNA during the PCR. The competitor was v-erbB DNA. The competitor was then sequenced to verify the incorporation of the gene-specific primers and was 400 bp long. The same primers were used to amplify 1 fg of competitor and scaled concentrations of cDNA. The log of cDNA/competitor was plotted against the target concentrations producing a coefficient r²>0.98 (data not shown). The same assay was developed for Fgf10. Competitive PCR quantification allows accurate assessment of mRNA levels and is reliable without contaminating DNA species.

Electrophoresis and densitometric analysis. Target and competitor PCR products were separated by size using electrophoresis in 3% agarose gels (NuSieve, FMC Bioproducts, Rockland, Me.). Gels were stained with 5 μg/ml of ethidium bromide and

photographed using a digital camera (Cohu, San Diego, Calif.). Band intensities were determined by densitometric analysis with ImageQuant band-analyzing software (Molecular Dynamics, Sunnyvale, Calif.). β-actin mRNA levels were measured in an identical fashion in both groups as an internal control for RNA extraction and CDNA production. All mRNA values were normalized to β-actin mRNA levels, which were the same for nitrofen-exposed and control lungs.

Statistical analysis. Morphometric data are reported as means +/− standard deviation. Densitometric data are reported as mean ratios of control values. Data from nitrofen-exposed lungs were compared to that of control lungs using a two-tailed Student's t test. Ap value of less than 0.05 was considered statistically significant.

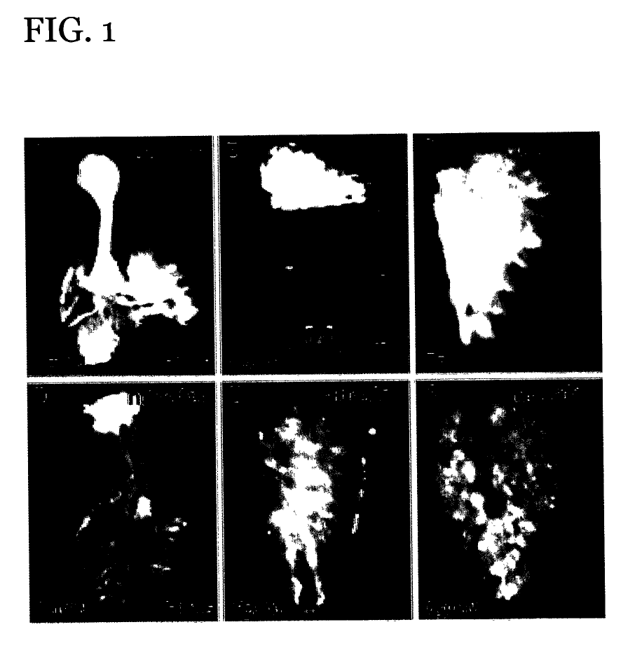

Results—Decreased spatio-temporal expression of Fgf10 in hypoplastic lungs. Fgf10 transcripts were studied during lung development by whole mount in situ hybridization. At E12.5 and through E15.5, high levels of Fgf10 expression were present in the mesenchyme adjacent to the epithelial buds of wild type lungs (FIG. 1A and IC), correlating with the sites of future dichotomous branching. In contrast, in Nitrofen-treated lungs, the temporospatial pattern expression of Fgf10 was markedly impaired (FIG. 1D). Interestingly, loss of Fgf10-expression appeared to correlate well with the severity of lung hypoplasia (FIG. 1B): Fgf10 transcripts could be detected only in the caudal part of the left lobe in moderately severe hypoplasia (FIG. 1F), whereas expression was nearly totally abolished in examples with more severe hypoplasia (FIG. 1E).

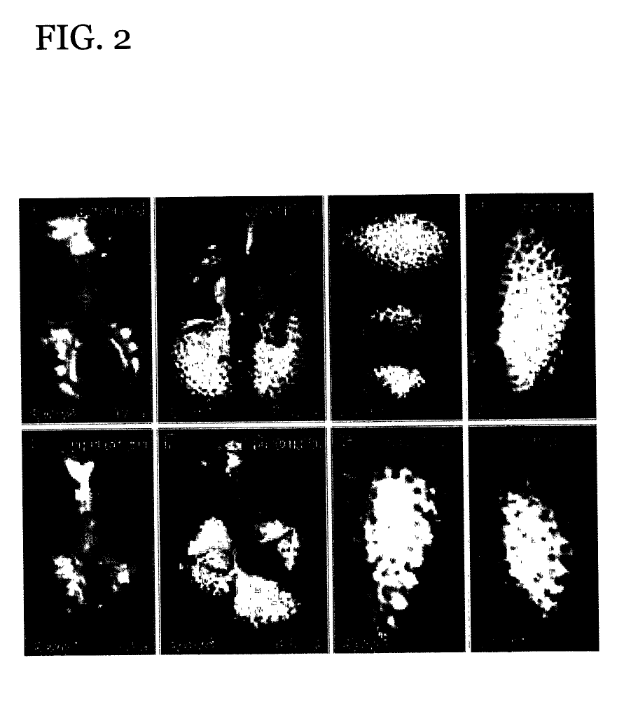

Whole mount in situ hybridization localized mSpry2 expression in the distal epithelium from E12.5 to E15.5 in normal lungs (FIG. 2A and 2B). In Nitrofen-exposed lungs mSpry2 seemed to be less expressed at E12.5 (FIG. 2A and 2E), but through E13.5 to 15.5 mSpry2 was expressed normally at the distal tips of epithelial buds (FIG. 2C, 2D, 2F-2H).

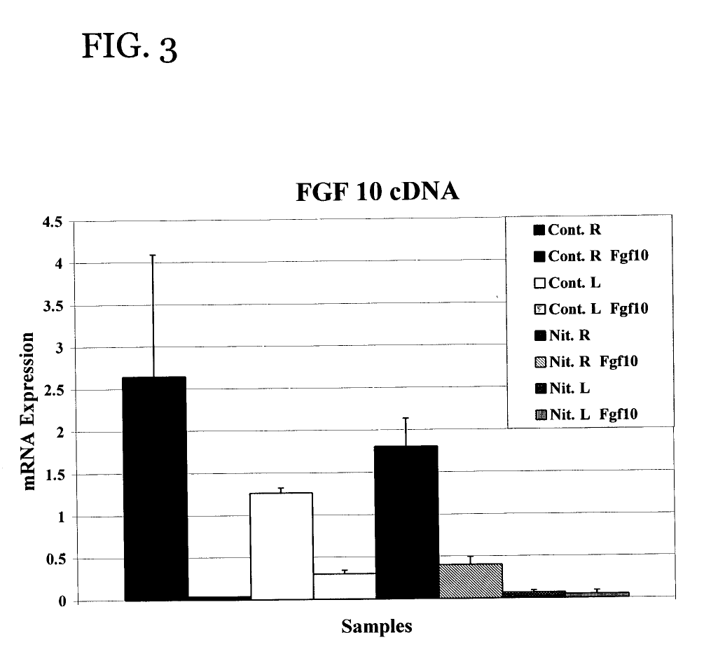

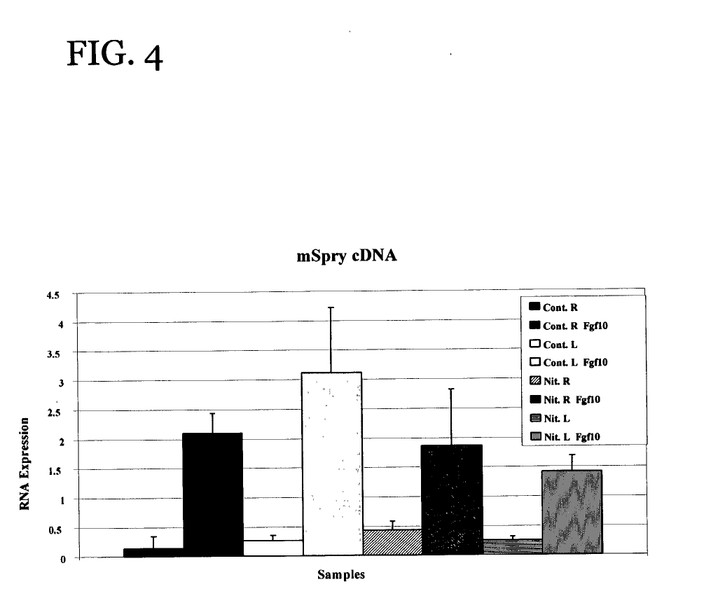

Nitrofen exposure, FGF10 and relative mRNA expression of Fgf-10 and mSpry2. Fgf10 and mSpry2 expression levels were measured by competitive RT/PCR and compared between wild type versus Nitrofen-exposed and right versus left E12 lungs after 4 days in culture. The results of this analysis are shown in FIGS. 3 and 4 and are all corrected as ratios to β-actin mRNA. The mean ratio of Fgf10 expression relative to β-actin was 2.65 in right and 1.26 in left wild type lungs. These relative levels of expression decreased respectively to 0.4 (right) and 0.3 (left) following exposure to exogenous FGF 10 (all p<0.05). It is also interesting to note that Fgf10 mRNA levels were reduced significantly following in-utero exposure to Nitrofen both in the right and left lungs. On the other hand, the mean level of expression of mSpry2 mRNA did not differ significantly between right versus left lungs in wild type or Nitrofen-exposed embryos. However, levels of mSpry2 expression did increase by 4- to 10-fold in the presence of exogenous Fgf10 under all conditions examined.

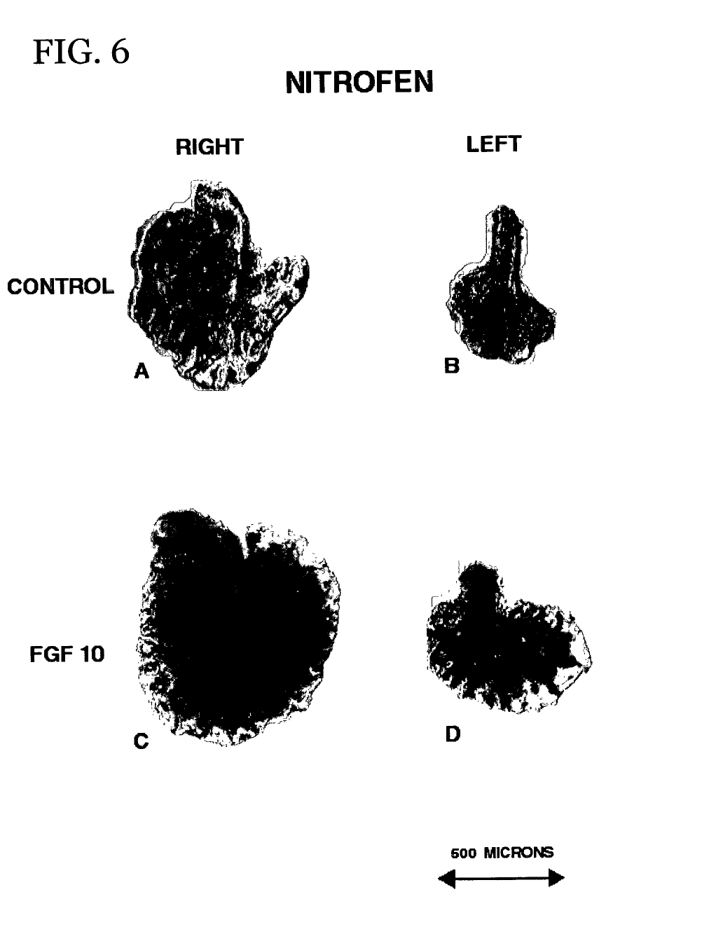

Nitrofen exposure, lung branching morphogenesis and FGF 10 rescue. Nitrofen exposure produced a profound decrease in branching morphogenesis, which was already evident at E12 and persisted when E12 lungs were cultured for 4 days, as illustrated in FIG. 6 and quantified in FIG. 3. Nitrofen exposure resulted in an almost complete arrest of lung budding in the left lung (see FIG. 6, panel B). The effects on branching in the right lung were also very striking, budding over 4 days in culture was very significantly decreased in the right lungs following Nitrofen exposure (See FIG. 6 panel A).

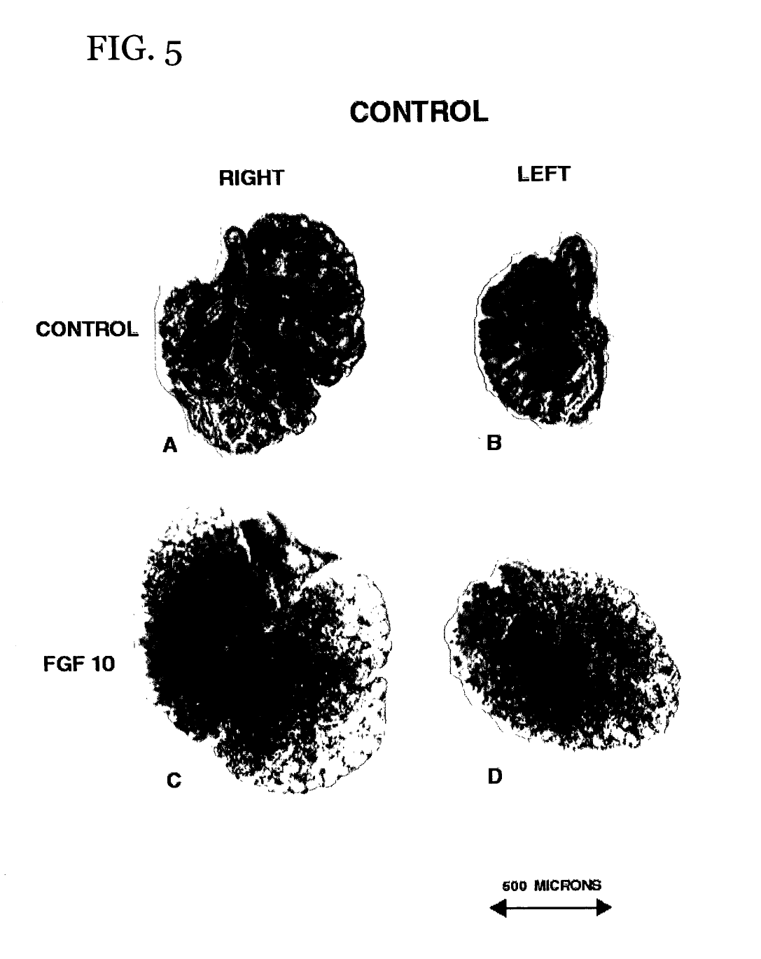

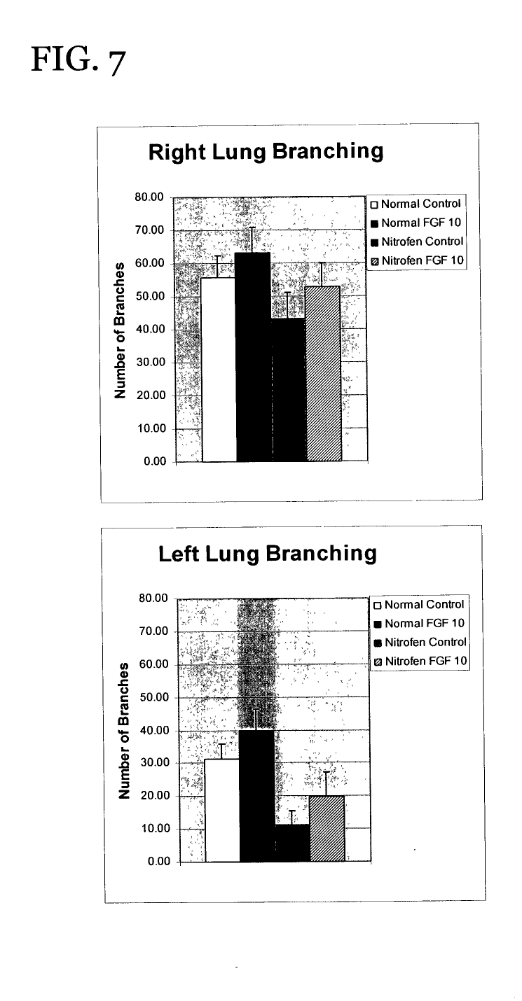

Exogenous FGF 10 produced a very striking and significant increase in overall size, lumen size, and branch numbers during morphogenesis in wild type control E12 lungs over 4 days in culture (see FIG. 5). FGF10 also produced a significant increase in size and complexity of E12 Nitrofen-exposed lungs over 4 days in culture (see FIG. 6). However, while the proportional increase in branch numbers was the same as in wild type, the final number of branches in nitrofen-exposed lungs was less than wild type, probably because of the relatively smaller starting number. However, the gains in branching with FGF 10 following Nitrofen-exposure were important, with the right lung restored to the same number of branches as wild type lungs grown without benefit of FGF10, while numbers of branches in the left lung increased by 77% (FIG. 7).

The numbers of control and Nitrofen exposed lungs placed in culture were as follows: control (Right n=25, Left n=20), control+FGF10 (500 ng/ml) (Right n=25, Left n=20) Nitrofen-exposed (Right n=25, Left n=15), Nitrofen-exposed+FGF10 (500 ng/ml) (Right n=25, Left n=20).

The temporospatial pattern of Fgf10 expression in wild type murine embryonic lung supports the concept that FGF10 plays a key role in directional outgrowth and possibly induction of epithelial buds. By using whole mount in situ hybridization and competitive RT/PCR, it is shown that Fgf10 mRNA expression is severely temporospatially disrupted and significantly decreased in Nitrofen-exposed lungs as compared to control lungs. In addition, exogenous FGF10 induces significant lung growth by stimulating lung branching morphogenesis in both control and Nitrofen-exposed lungs in culture. These observations suggest that abnormal FGF10 signaling may account substantially for decreased branching in Nitrofen-induced lung hypoplasia before the onset of CDH.

The most attractive pieces of data supporting a unifying theory that Nitrofen-induced lung hypoplasia is caused by abnormalities of Fgf10 expression are as follows:

(i) Fgf10 signaling is clearly necessary for lung morphogenesis distal to the trachea, since null mutation of Fgf10 completely abrogates this process (Min et al. (1998) Genes Dev 12:3156-61).

(ii) Many of the associated tracheobronchial and skeletal abnormalities caused by nitrofen exposure can also result from abnormalities in the fibroblast growth factor receptor signaling pathway, including skull hypoplasia, axial appendicular and rib anomalies (Migliazza et al. (1999) J Pediatr Surg 34:1624-9; Xia et al. (1999) Pediatr Surg Int 15:184-7).

(iii) The findings reported herein, that exogenous FGF10 both stimulates wild type lung morphogenesis and substantially rescues Nitrofen-induced embryonic lung hypoplasia in culture further support the concept that Nitrofen may interfere with Fgf10 expression.

### EXAMPLE 2

Telomerase in Alveolar Epithelial Development and Repair

Materials and Methods. Preparation of lung tissue and analysis by immunohistochemistry. Analysis of mTERT expression was performed on lung sections from embryonic and neonatal mice, and on sections from control and hyperoxia treated adult rats. Tissue was fixed in 4% paraformaldehyde in PBS, dehydrated and embedded in paraffin according to the method described by Tesarollo and Parada ((1995) Methods in Enzymology 254:419-429). Tissue was sectioned, re-hydrated, and subjected to immunostaining using a mouse and rat cross-reactive antibody to hTERT (Santa Cruz Biotechnologies). Antigen positive cells were detected using reagents from the Histostain Plus kit from Zymed, with amino-ethyl carbazole (AEC) as the chromagen. Sections were observed and photographed using an Olympus Light microscope. Immunocytochemical analysis of cultured cells followed essentially the same protocol. Cells were fixed for 15 min in 4% paraformaldehyde in PBS, then stained according to manufacturer's instructions, using the Zymed kit. The PCNA antibody used in this experiment was from Santa Cruz Biotechnologies.

TRAP assay. Sample preparation and TRAP assays were performed according to the TRAP-EZE protocol (Oncor). Briefly, at least 10⁶ cells for each sample were lysed in IX CHAPS lysis buffer. The lysate was clarified by centrifugation, and protein content was measured using a modified Coomassie binding reagent (Bio-Rad). In order to assay telomerase activity, samples were incubated with a [γ-³²P] dATP end-labeled telomerase-specific primer at 30° C. for 30 min for telomere primer extension. The telomerase products were then amplified by 30 rounds of two-step PCR (94° C./30 sec, 60° C./30 see). The samples were subjected to 12.5% non-denaturing polyacrylamide gel electrophoresis (PAGE) in 0.5X TBE buffer (45mM Tris-Borate, 1 mM EDTA) for 1 hr at 500V. Gels were dried and exposed to X-ray film in order to visualize the telomerase products. Each assay included a positive control in the form of lysate from telomerase positive A549 lung adenocarcinoma cells, as well as a PCR internal amplification control, provided by Oncor, and a PCR contamination control lane, consisting of all sample elements with the exception of cell lysate. All cell samples were individually controlled for non-specific PCR products by inclusion of a heat inactivation control, for which identical aliquots of each sample were incubated at 85° C. for 10 min in order to inactivate telomerase.

Hyperoxia treatment and adult and fetal AEC2 culture. Adult male Sprague-Dawley rats were exposed to short-term hyperoxia as described previously (Bui, et al. (1995) Am. J Physiol. 268 (Lung Cell. Mol. Physiol. 12): L262-L635). Briefly, rats were placed in a 90 cm×42 cm×38 cm Plexiglas chamber, and exposed to humidified >90% oxygen for 48 hours, then allowed to recover in room air for 48 hours. Control rats were kept in room air during the treatment period. At the end of the exposure/recovery period, the animals were anesthetized by I.P. injection of pentobarbital. Following complete exsanguination by normal saline perfusion via the pulmonary artery, lungs were lavaged to remove macrophages, then subjected to elastase digestion for isolation of AEC2. Differential adherence on IgG plates was used to eliminate non-AEC2 cells from the preparation (Dobbs, et al. (1986) Am. Rev. Resp. Dis. 134:141-145). E21 fetal (saccular stage) rat AEC2 were obtained by trypsin digestion of whole lungs and differential plating, according to the method of Jassal et al. ((1991) In Vitro Cell. Dev. Biol. 27A:625-632). Timed-pregnant animals were euthanized by chloroform inhalation and fetuses were weighed in order to confirm gestational age. For both fetal and adult AEC2 culture, cells were plated at 2×10⁵ cells/cm² in DMEM with 10% FBS plus antibiotics for 24 hr, then harvested by trypsinization for TRAP assay preparation, or fixed in situ for immunohistochemical analysis. Immunostaining of attached cells isolated by these methods with an anti-SP-C antibody confirmed that >95% of the attached cells were SP-C positive AEC2 (Bui, et al. (1995) Am. J Physiol. 268 (Lung Cell. Mol. Physiol. 12): L262-L635).

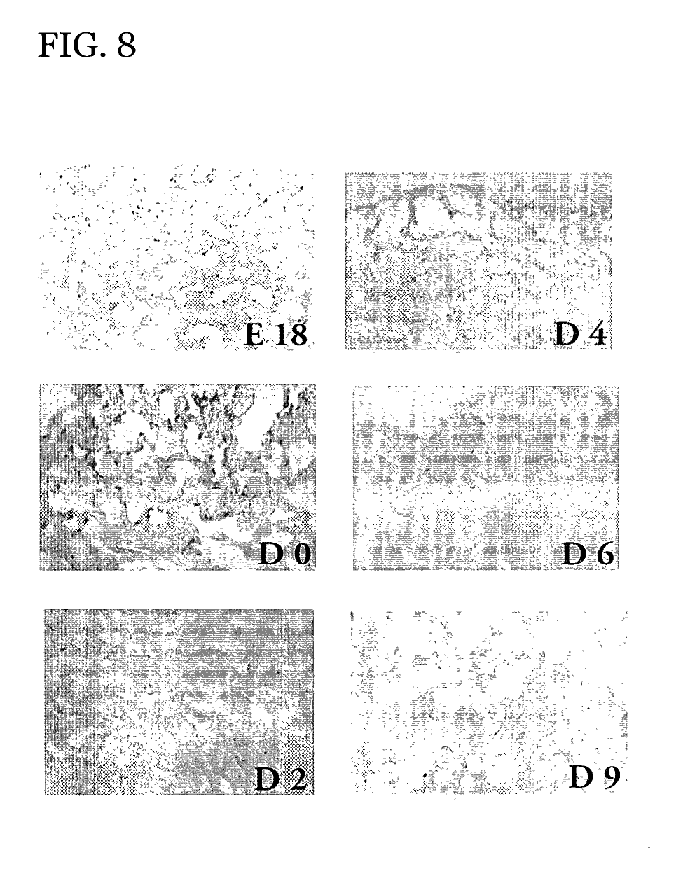

Results. Telomerase expression is restricted to a subpopulation of mouse lung epithelial cells through embryonic development, and is down regulated following birth. Lung sections from staged mouse embryos were fixed, paraffin embedded and sectioned, then immunostained using an antibody raised against the catalytic subunit of human telomerase, hTERT, which cross-reacts with both mouse and rat TERT. Whole lungs were obtained from embryos at gestational age E18 (FIG. 8, E 18), and from neonates at one hour post-birth (D 0), and at two days (D 2), four days (D 4), six days (D 6), and nine days (D 9) following birth. Scattered epithelial and mesenchymal staining in lungs of mouse embryos from gestational age E18 through post birth day 6 was found. Epithelial expression appeared strongest at E18 through the day of birth, with staining confined to individual cells. Expression appeared to peak at this time, then declined over the next nine days. During this period, the generalized expression pattern became restricted to discrete patches near the external surface of the lung (FIG. 8, D 6). By day nine, telomerase expression was almost undetectable. A similar lack of mTERT expression was observed by immunostaining adult mouse lung epithelium (data not shown).

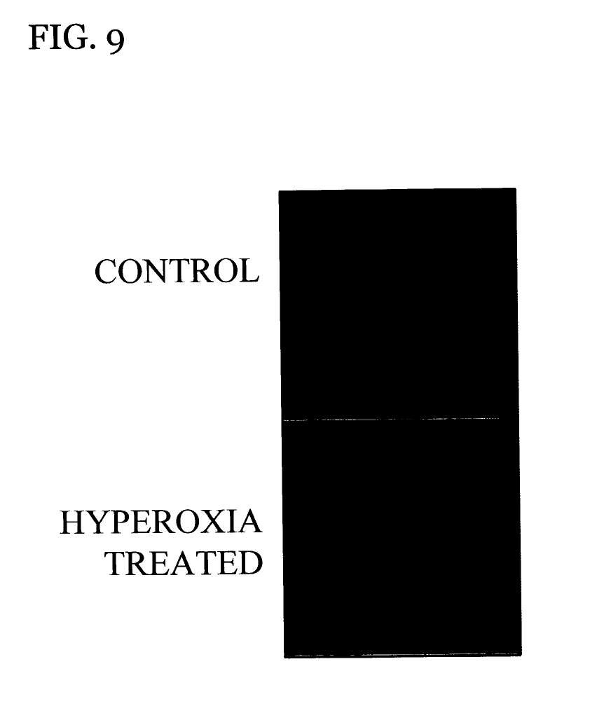

Telomerase expression in adult lung is induced during the repair phase following hyperoxic injury. Previous studies showed that exposure of animals to hyperoxia induces a proliferative response in normally quiescent lung tissue as part of a process of repair. In order to determine if re-induction of telomerase expression was a part of this process, fixed, paraffin-embedded sections were obtained from the lungs of adult rats treated with hyperoxia for 48 hours, then allowed to recover in room air for various periods of time. Lung sections from age- and weight-matched animals, which breathed room air throughout the treatment and recovery period, were used as controls. Sections were immunostained as described for embryonic and neonatal mouse lung sections, using the same anti-TERT antibody. Analysis showed that, as with adult mice, negligible TERT expression could be detected in control adult rat lung epithelium (FIG. 9, top panel). In contrast, TERT expression increased dramatically in the lung tissue of animals subjected to hyperoxia for 48 hours, then allowed to recover in room air for 48 hours (FIG. 9, bottom panel). While scattered TERT expression was observed at 0 and 120 hours recovery following 48 hours of hyperoxia treatment (data not shown), peak telomerase expression occurred during the period 48 hours following treatment. Previous studies showed that this is also the time period where maximum proliferation of AEC2 following injury occurs (Buckley et al. (1998) Amer. J. Physiol. 274 (Lung Cell. Mol. Biol.):L714-720; Bui, et al. (1995) Am. J PhysioL 268 (Lung Cell. Mol. Physiol. 12): L262-L635). These data suggest that activation of telomerase expression may coincide with proliferation and activation of a stem or progenitor cell population, which participates in re-population of the damaged lung epithelium during recovery from injury. Alternatively, the high percentage of telomerase positive cells observed during the repair phase may represent a hyperoxia-resistant population, enriched by the loss of hyperoxia-sensitive cells during the injury period. Further experiments will be required to differentiate between these two possibilities, or to determine if the telomerase expression profile of lung tissue following injury is due to a combination of both phenomena.

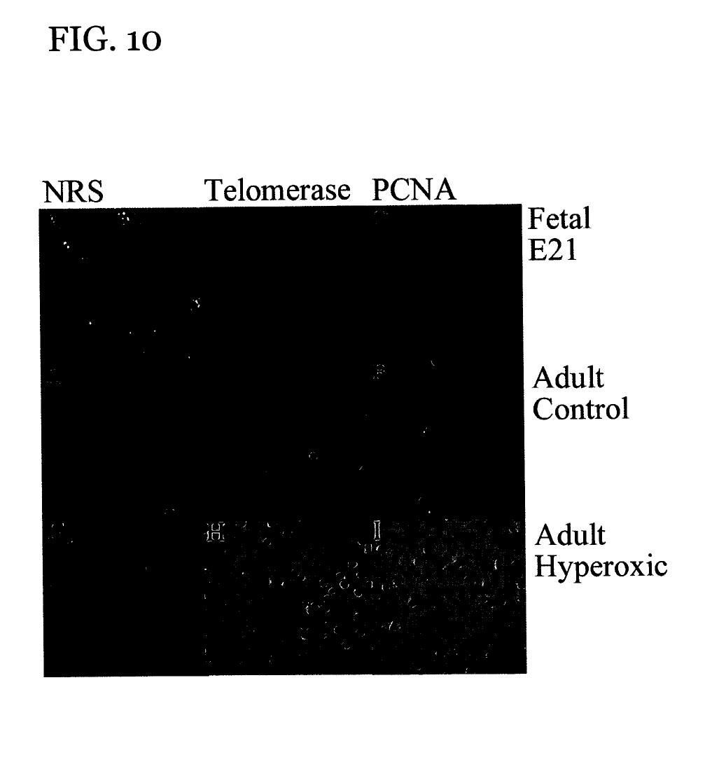

Telomerase expression in fetal and adult AEC is correlated with proliferative status. In situ immunostaining of whole lung tissue, as described above, indicated that particular cells in developing and repairing lung expressed telomerase, though the identity of those cells could not be determined. It has been postulated that the alveolar epithelial type 2 cell serves as the source of the re-epithelialization in repairing lung, and performs the same function during the later stages of development. It was speculated that cells performing these functions would by necessity express telomerase, since they would be undergoing multiple rounds of cell division during tboth development and the repair process. AEC2 were isolated from gestational age E21 rat embryos according to the method of Jassal, et al. ((1991) In Vitro Cell. Dev. Biol. 27A:625-63). Fetal rat lungs are at the saccular stage of development within this time period, and the rat E21 stage was chosen in order to compare to the mouse E 18.5 stage, where, by immunostaining, high mTERT expression was observed. AEC2 from adult rats subjected to a hyperoxic environment for 48 hours, then allowed to recover in room air for 48 hours, as well as control animals who breathed room air for the entire period, were also isolated, using the method described by Dobbs, et al. ((1986) Am. Rev. Resp. Dis. 134:141-145). Both adult and fetal cells were plated on plastic in complete medium for 24 hours. At that time, duplicate cultures were analyzed for telomerase and proliferating-cell nuclear antigen (PCNA) expression by immunostaining. The same anti-telomerase antibody and an adaptation of the protocol used for tissue section immunocytochemistry, as described in FIG. 8, were used for this purpose. The data obtained by these analyses showed that expression of telomerase was high in the majority of E21 rat AEC, though this expression was not completely uniform. A similar pattern of expression was observed using an antibody to the proliferation specific protein, PCNA (FIG. 10, Panels B and C). In contrast, telomerase expression in the adult control sample was observed in very few cells (FIG. 10, Panel E). Surprisingly, a very low level of PCNA expression could also be detected in these cells, perhaps indicating a transient proliferative response to isolation and culture (FIG. 10, Panel F). Exposure to, and recovery from, hyperoxia in vivo resulted in re-expression of telomerase at high levels in the adult population, in a pattern similar to that observed in the fetal population, in that expression levels varied from cell to cell (FIG. 10, Panel H). These cells also exhibit a uniform increase in PCNA expression, indicating a strong, proliferative profile (FIG. 10, Panel I). Thus, telomerase expression was confined in the adult AEC to a portion (albeit, in response to hyperoxia, a large one) of the total population. Specificity of expression for the telomerase antibody was confirmed by use of normal rabbit serum in place of primary antibody (FIG. 10, Panels A, D, and G).

Telomerase activity is observed in fetal AEC2, and can be re-induced in adult AEC2 following hyperoxic injury. In order to determine if the TERT expression observed in fetal and adult lung tissue could be correlated with telomerase activity in lung epithelial cells, a portion of the primary cultures of AEC2, isolated from fetal and adult rats, was harvested and analyzed for endogenous telomerase activity by use of the PCR-based TRAP assay. For each sample, radioactively labeled telomerase end products (telomeric repeats) were subjected to TBE-PAGE, and the results were detected by radiography. The number of telomeric repeats produced by each sample was counted in order to give a relative estimate of telomerase activity contained within each sample, which contained 80 ng of each cell lysate. Using a ratio of 10 ng protein per cell, telomerase activity observed in each sample was taken to correspond to the activity present in approximately 8 AEC2. Though these isolated AEC2 populations are, by SP-C staining, 95% pure (Bui, et al. (1995) Am. J Physiol. 268 (Lung Cell. Mol. Physiol. 12): L262-L635), the minor fibroblast contamination which is still present in primary cultures could account for some of the observed telomerase activity. Therefore, the number of cell equivalents used for each assay was kept at this minimum level, to insure that the sample contained lysate enriched to a high degree with epithelial cell components, and that fibroblast contribution to the assay remained negligible.

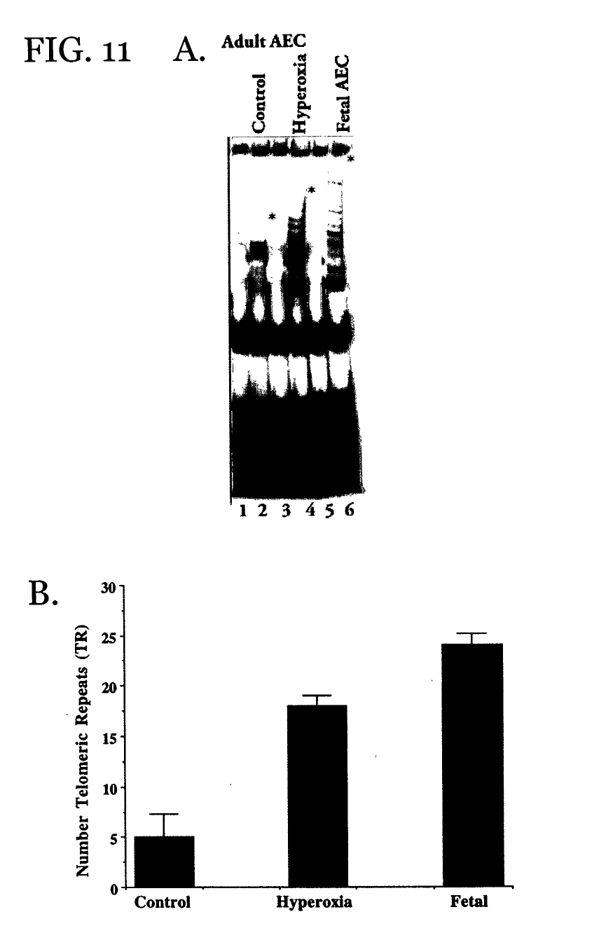

The results of the TRAP assay showed that telomerase activity could be correlated with telomerase expression as assayed by immunostaining during both development and injury repair. While the level of telomerase activity in the E21 fetal AEC2 was quite high (FIG. 11A, lane 6), the level in the same number of cells obtained from a six-week old adult male rat was much lower (FIG. 11A, lane 2). Interestingly, AEC2 obtained from adult animals that had been exposed to a hyperoxic environment showed a significant increase in telomerase activity (FIG. 11A, lane 4). This result was observed reproducibly, and FIG. 11B shows a quantitation of the TRAP assays performed on a number of animals (values are mean +/−SD, n=3 for fetal, 6 for adult animals). The average number of telomeric repeats (TR) produced by each type of sample was correlated to ladder bands by the formula TR=n+3, where n=the number of bands in each ladder, and TR is taken in this assay as a reflection of relative telomerase activity. By this calculation, the fetal sample produced the largest average number of telomeric repeats: twenty-four. The sample from the control animal contained the fewest average number of telomeric repeats, five, as would be expected from cells obtained from highly differentiated tissue. The number of repeats produced by lysates of cells from animals treated with oxygen showed a consistent increase, reflecting increased telomerase activity. The average number of telomeric repeats produced by hyperoxic adult AEC2 was eighteen. Using Student's t-test, the differences in the TR between control and hyperoxic (*P<0.005) and between control adult and fetal (**P<0.001) cells were determined to be significant.

The data presented here demonstrate that telomerase, the polymerase responsible for telomere maintenance and extended cellular life span, is expressed in developing rodent lung, then down-regulated after birth. It has been established by Greenberg and colleagues (Greenberg, et al. (1998) Oncogene 16:1723-1730) that mTERT mRNA expression peaks in the whole embryo at mid-gestation (E9.5 through E15.5) during mouse development. Interestingly, the mTERT transcript is maintained at a very low level when compared to that of a housekeeping gene, such as actin. However, expression is broadly distributed through many organs, including lung. The transcript level in the lung of newborn animals is intermediate between the high levels observed in organs with proliferative indices (intestine, testes) and the low levels in organs composed of less proliferative tissues (brain, heart). This pattern is maintained in adult animals, indicating that whole mouse lung may require a certain basal level of telomerase expression for proper function. However, the identity of the telomerase expressing cells in the lung was not described by Greenberg, who used whole lung lysates as a source of TERT mRNA. It is reported here that the expression of the mTERT catalytic subunit, as assayed by immunostaining of sections of lungs harvested from staged mouse embryos and neonates, is restricted to a subpopulation of cells within the alveolar epithelium. In mice, expression levels peak at El 8.5, just prior to birth, then decrease over the period of alveolarization, at 4 to 6 days after birth. By 9 days following birth, expression of the mTERT protein is almost undetectable. As expected, mTERT expression is also low in adult lung from normal animals.

The in situ results observed here demonstrate that the percentage of cells which express telomerase is higher in the repairing adult lung than the percentage observed in developing lung tissue, though the percentages of positive cells in the isolated, AEC enriched populations in culture is similar. This discrepancy may simply reflect that the percentage of AEC2 in the developing lung is much smaller than that observed in the adult lung, and that isolation of an AEC rich population pools together all the telomerase positive lung cells from each source. The large numbers of telomerase-positive cells in the AEC population isolated from repairing lung could represent those cells which have repopulated the damaged tissue during the injury and recovery periods, and which may soon exit the proliferative pool in order to take up AEC2 differentiated functions.

The foregoing is illustrative of the present invention, and is not to be construed as limiting thereof. The invention is defined by the following claims, with equivalents of the claims to be included therein.

That which is claimed is:

1. A method of stimulating the growth of lung alveolar surface in a lung in need thereof, comprising:
providing progenitor or stem cells capable of regenerating lung alveolar surface; and
administering said progenitor or stem cells to said lung in an amount sufficient to stimulate the growth of lung alveolar surface therein.

2. A method according to claim 1, wherein said lung is in vivo in a subject in need of said treatment.

3. A method according to claim 1, wherein said lung is ex vivo, and wherein said administering step is followed by the step of:
transplanting said lung into a recipient in need thereof.

4. A method according to claim 1, wherein said subject is a mammalian subject.

5. A method according to claim 1, wherein said subject is a human subject.

6. A method according to claim 1, wherein said step or progenitor cells are from the same species as said subject.

7. A method according to claim 1, wherein said progenitor cells are autologous cells.

8. A method according to claim 1, wherein said administering step is carried out by intravenous injection, intra-arterial injection, or intra-bronchial administration.

9. A method according to claim 1, wherein said stem or progenitor cells are lung cells.

10. A method according to claim 1, wherein said stem or progenitor cells are bone marrow cells.

11. A method according to claim 1, wherein said stem or progenitor cells are embryonic stem cells.

12. A method according to claim 11, wherein said embryonic stem cells contain a cell nucleus that is autologous to said subject.


SEQUENCE LISTING (2 sequences)

SEQ ID NO: 1 - DNA, 21 bp, synthetic construct
  misc_feature (1)..(21): Synthetic Oligonucleotide Primer
tgtgaggactgtggcaagtgc

SEQ ID NO: 2 - DNA, 20 bp, synthetic construct
  misc_feature (1)..(20): Synthethic Oligonucleotide Primer
tttaaggcaacccttgctgg